(19) United States
(12) Patent Application Publication
(10) Pub. No.: US 2013/0178691 A1
(43) Pub. Date: Jul. 11, 2013

(54) Electrically lossy magnetic liquid composition for controlled tissue heating

(71) Applicants: Petr Saha, Mladcova-Zlin (CZ); Natalia Kazantseva, Moskva (RU); Petr Humpolicek, Bystrice pod Hostynem (CZ); Robert Moucka, Zlin (CZ); Jarmita Vilcakova, Zlin (CZ); Pavel Valasek, Uherske Hradiste (CZ)

(72) Inventors: Petr Saha, Mladcova-Zlin (CZ); Natalia Kazantseva, Moskva (RU); Petr Humpolicek, Bystrice pod Hostynem (CZ); Robert Moucka, Zlin (CZ); Jarmita Vilcakova, Zlin (CZ); Pavel Valasek, Uherske Hradiste (CZ)

(21) Appl. No.: 13/348,327

(22) Filed: Jan. 11, 2012

(51) Int. Cl. A61N 2/00; B32B 15/02; B82Y 99/00

(52) U.S. Cl. 600/10; 428/407; 977/773

(57) ABSTRACT

A dental floss apparatus comprised of a pair of handles that are configured for engagement with the fingers with strands of flossing substrate extending therebetween. The flossing substrates are spaced from each other, running substantially parallel and extending within an opening formed between the ends of the handles. A dissolvable substrate is positioned within this opening through an engagement with one or both of the handles or the flossing substrate, and upon contact with the surface of the teeth, dissolves, and deposits a residue on the surface of the teeth and the surrounding area of the mouth. The dissolvable substrate may be formed of two substrate pieces with a central gap between them defining a means for targeting insertion of the substrate between adjacent teeth and may be colorized or luminescent and have an additive material such as mouthwash, breath freshener, cooling agent, or flavor.

### BACKGROUND OF THE INVENTION

1. Field of the Invention

The invention relates to the use of nanomaterials forming magnetic liquid compositions applicable for controlled tissue heating and being useful preferably as a mediator in RF hyperthermia with a goal to provide selective heating of tumors.

2. Description of the Related Art

Today, tissue heating which is preferably known as hyperthermia and used as a medical heat treatment of tumors is well established. The main difficulty of the said procedure is that the effective temperature range in hyperthermia is very narrow: from 42° to 45° Celsius, and in practice it is difficult to achieve temperature higher than 43° C. At lower temperatures, the effect is minimal. At temperatures exceeding 45° C., normal cells are damaged. Treatment at temperatures between 42° C. and 45° C. is cytotoxic for cells in an environment with deficiency of O2 and low pH, conditions that are found specifically within tumor tissue, due to insufficient blood perfusion; whereas most normal tissues are able to endure treatment for 1 hour at a temperature up to 44° C. without any negative impact. The molecular mechanisms for cell death at temperatures above 42° C. are understood now, with protein denaturation being the main mechanism among these. Simultaneously, such temperatures cause an increase in sensitivity of tumor cells to radiation and chemotherapy. Increased response and survival rates of cancer patients clearly indicates increased efficiency of radiotherapy and chemotherapy in combination with hyperthermia as compared with radiotherapy and chemotherapy alone. Thus, it is widely accepted that the main benefit of hyperthermia is improving the results of the conventional treatment strategies within a framework of multi-modal treatment concept: chemotherapy and radiotherapy combined with a hyperthermia.

Hyperthermia has become standard in the oncology practice due to the development of heating techniques based on energy delivery by electromagnetic waves through either ohmic heating by radio frequency currents (0.1-300 MHz), or heating by means of microwave electromagnetic radiation (300-3000 MHz). The existing hyperthermia applicators are based on the following methods: (i) impedance, inductive and capacitive heating in kHz and MHz regions; (ii) antenna (phase) array in 100 MHz region; (iii) microwave radiation over 200 MHz. The most commonly used frequencies in hyperthermia are 13.56 MHz, 27.12 MHz, 433.915 MHz and 2450 MHz, which are designated ISM (industrial, scientific, and medical) frequencies in the U.S. and Europe.

Due to the narrow temperature range of hyperthermia, the response rate of the tumor is highly dependent on how much of it is heated to therapeutic level. Therefore, in RF hyperthermia, the final temperature of tumors mainly depends on energy deposition and its localization. When electromagnetic heating methods are used, the energy deposition is a complex function of the frequency, intensity, and polarization of the applied fields, the applicators size and geometry, as well as the size, depth, geometry, and dielectric properties of the tumor.

In order to overcome the aforesaid problems of heat delivery by electromagnetic waves, apart from innovation in device and treatment planning models in hyperthermia, the localization of heating by radio absorbing materials embedded into the tumor is being intensively studied. This kind of hyperthermia is based on the presence of magnetically and/or electrically lossy material, so called mediator, which absorbs and subsequently dissipates energy of electromagnetic radiation into heat resulting in selective heat treatment of tumors on the microscopic level. This method is promising especially for delivering thermal energy for deep seated solid tumors.

The concept of mediator usage in hyperthermia was firstly described by Gilchrist in 1957. In this study, magnetite particles of diameter 0.02-0.1 μm were used for treatment of lymph nodes of a dog in vivo under exposure to alternating (ac) magnetic field of strength 200-470 Oe. It was found that a concentration of 5 mg of magnetite per gram of lymph node tissue and 3 minutes of heating at alternating magnetic field of strength 470 Oe leads to the total necrosis of the nodes. This type of hyperthermia became known as Magnetic Mediated Hyperthermia (MMH). Since the 1960s, MMH has developed three different pathways, all having in general the same methodology: the deposition of ferromagnetic material within or adjacent to tumor tissue followed by exposure to ac magnetic field. Today's advances in preparation of magnetic nanoparticles and improved physical understanding of their interaction with external electromagnetic field, as well as with the physiological environment has led to the development of the clinical alternating magnetic field applicator system (Hyperthermiesysteme GmbH, Berlin, Germany) operating at a frequency of 1 MHz with a field amplitude up to 15 kA/m. However, it seems that MMH will not be widely used in clinical practice in near future for several reasons, namely: (i) the lack of magnetic materials with superior magnetic properties and simultaneously suitable for in vivo applications; (ii) the deficiency in production of the AC magnetic field applicator system and its cost.

Appropriate materials for MMH should have specific loss power (specific magnetic/dielectric power dissipation in a medium) that would allow prompt heating of the tumor up to 43° C. (the ideal temperature for apoptosis of tumor cells without harming adjacent normal cells) in an ac magnetic field with amplitude (H) and frequency (f) which do not violate the safety standards for human (H≦15 kA/m; 0.05≦f≦1.5 MHz). Consequently, the first strategic objective in MMH is to tailor the properties of the well-known magnetic iron oxides, e.g. magnetite or maghemite that has been already approved for human use and optimize its size and shape dependent magnetic characteristics, and functionalize them suitably with a view to assess appropriate cytotoxicity and nanoparticle uptake by tumor tissue. Recently many patents focusing on the methods of obtaining various types of non toxic magnetic nanoparticles having a size suitable for use (generally between 100 and 300 nm), a restricted size distribution and the ability to remain stable in a physiological environment have been published. The highest specific loss power (SLP), nearly 1000 W/g at 410 kHz and 10 kA/m, was obtained for magnetosomes, however it still falls short of required value of SLP, considered to be 10 kW/g.

As noted above, the lack of mass production of the AC magnetic field applicator systems is one of the reasons that hinder the realization of the MMH in clinical practice. However, capacitive heating of tumors using a RF electric field has been adopted in practice worldwide. Examples of commonly employed RF-capacitive heating applicator systems are Thermotron RF-IV, and SYNCHROTHERM RF and Yacht-5.

A typical treatment using RF capacitive hyperthermia (RFCH) consists of two 30 to 60 minute sessions a week, for six to eight sessions. For patients, such a long period of heating is a severe burden. To make a capacitive heating session shorter, Kobayashi, with his group, suggested using magnetic particles likewise the methodology adopted in MMH [A. Ito and T. Kobayashi, Intracellular hyperthermia using magnetic nanoparticles: a novel method for hyperthermia clinical applications, Therm Med, 2008, 24(4): p. 113-129]. They demonstrated the enhancement of hyperthermic effect by magnetic nanoparticles and temperature profile in RF capacitive heating in vivo. Magnetite cationic liposomes (MCLs) were injected into a tumor on the femur of a rat and 8 MHz-RF capacitive heating was applied to the rat under ‘mild heating’ conditions (power output at 60 W for 5 minutes and then at 40 W for 40 minutes) to reach the tumor temperature of about 43°-44° C. As a result, complete tumor suppression was observed in 71% of MCLs-injected rats. The histological observation of the removed tissue showed wider necrotic area in the case of MCLs-injected tumor than that in MCLs-free tumor. This was explained by the enhanced dissipation of electromagnetic energy in the tumor region associated with dielectric losses of magnetite particles.

Generally, electrically lossy materials can be used as mediators with a view to control the heating of the tumor under the exposure to alternating electric fields in RFCH, or under the exposure to electromagnetic field in the Radiative hyperthermia. Both means of energy delivery can work with different materials, whether magnetic or nonmagnetic.

Patents US 07122030B2 and US 00763229B2 propose a method of localized and temperature controlled tissue heating, comprising the introduction of ferroelectric materials with Curie temperature between 42° and 50° C. in targeted region of patient and applying ac electrical field. Ferroelectric materials consist of 15 μm-mean-sized particles coated by bio-compatible and bio-degradable polymers which are dispersed in a pharmaceutically suitable solution. This material heats via hysteresis losses up to Curie temperature under applied electrical field, making it possible to heat ferroelectric particles using radiation received from time-varying electrical field radiated from a suitably driven radiofrequency antenna or generated between the plates of a capacitor driven by a time-varying wave form. The frequency provided by generator is selected in accordance with a sufficient penetration depth of radiation into human tissue, namely from 100 kHz to about 100 MHz. The essential applied power is selected between 20 W and 30 W.

Patent WO 2009/013630 A2 provides a Radiative Hyperthermia device which can be used together with multi-component nanoparticles, for example, noble-metal nanoparticles or magnetic nanoparticles coated by noble metals. These particles, additionally functionalized via dipolar interaction, can be heated up under the action of RF electromagnetic field due to the various mechanisms, namely hysteresis, magnetic relaxation or induced currents. Herein, RF electromagnetic field produced by the generator has controllable intensity and frequency, namely from 100 kHz to about 4 GHz.

Patent US 007510555B2 introduces a modified RF-capacitive heating device and method for proper focusing of electromagnetic energy on the malignant area. To achieve a deep energy delivery, prior to treatment “RF enhancers” should be introduced into the targeted area by any appropriate method. These “RF enhancers” are nanoparticles of conductive metals (iron, gold, etc.), which are either dispersed in various solutions (water, saline, etc.), or bonded to the biomolecules (antibodies, proteins, DNA, RNA, etc.). Depending on the material type, they can be intravenously injected, or delivered through particle targeting. The novelty of proposed method is to design mediator/device combination that makes possible to associate the operating frequencies of RF applicator with the resonant frequencies of the “RF enhancers”. To this end, the RF signal is modulated through amplitude or phase to obtain the frequency spectrum around the basic frequency (13.56 MHz and 27.12 MHz).

The main disadvantage of abovementioned patents (US007122030B2; US00763229B2; WO 2009/013630 A2; US007510555B2) is that the efficiency of mediator usage with a view to enhance the modality of RFCH and Radiative hyperthermia is estimated only indirectly on the basis of SLP versus RF field amplitude measurements at certain frequencies, neither in vitro nor in vivo results are included. Therefore, the relevance of concrete examples to real use may be in certain cases rather questionable and thus differ from clinical practice.

The problem to be solved by the invention is to develop mediator/RF-capacitive-heating-applicator combination that makes this method site-specific and thus minimizes exposure time and output power of hyperthermia session and minimizes negative impact on health tissue. Present invention is in its nature rather close to the material and method performed by Kobayashi and his group, that is why it can be taken as a closest state of the art.

### SUMMARY OF THE INVENTION

Based on the analysis of the efficiency of materials with magnetic and dielectric losses in hyperthermia, one can conclude the following:

RF hyperthermia for tumor therapy using mediators in the form of magnetically and/or electrically lossy materials has the potential for targeting tumor tissue selectively. However, there are a number of problems which make the employment of mediators in hyperthermia in clinical practice rather difficult. The main issue is that the dielectric and magnetic losses, and hence a SLP of existing nanomaterials is insufficient and therefore must be considerably increased for achieving useful therapeutic temperatures. Furthermore, SLP peak should match an operating frequency range of actual hyperthermia device. Additionally, due to a wide distribution of RF capacitive applicators in clinical practice, in order to make capacitive heating more selective for tumor tissue, the development of suitable mediators is current issue.

Thus the principal objective of the present invention is to provide a mediator with high dielectric losses at basic frequency(ies) of actual RF-capacitive hyperthermia device and thus make method site-specific under ‘mild’ heating conditions. This objective is attained by using of the electrically lossy magnetic liquid applicable for controlled tissue heating, preferably for tumor therapy by RF-hyperthermia, according to the invention. The said magnetic liquid comprises of electrically conductive component formed by magnetic nanoparticles with core-shell structure where core size is between 10-15 nm and polymer shell thickness is between 1-3 nm; the said component is capable of subsequent dissipation of energy of electromagnetic radiation into heat resulting in selective and controlled heat treatment of defined extent of the tissue. Its core-shell structure is based on iron oxide-containing cores organized in chains-like structures and capable to generate heat by applying alternating electrical field due to dielectric losses.

A method of using of the magnetic liquid according to the invention comprises:

(a) accumulation of electrically lossy magnetic liquid in the tissue, the said liquid comprises chain-like structures, formed by iron-oxide core-shell nanoparticles and causing high dielectric losses of magnetic liquid under exposure to alternating electric field at the frequencies commonly employed in RF-capacitive hyperthermia, preferably at 13.56 MHz and 27.12 MHz;
(b) application of alternating electrical field to generate heat in the tissue by excitation of electrically lossy magnetic liquid, thereby causing hyperthermia of the tissue.

A mediator(s), comprising magnetic nanoparticles and a matrix, wherein said particulate composition(s) combines following features: (i) dielectric losses high enough for subsequent dissipation of electromagnetic radiation energy into heat; (ii) magnetic nanoparticles have a core-shell-like structure with a core size of about 10-100 nm and a shell thickness of about 1-3 nm; (iii) magnetic nanoparticles are dispersed in biocompatible liquid.

According to the present invention it has been found that such electrically lossy particulate composition can be obtained in the form of a liquid comprising iron oxide nanoparticles of about 10 nm in situ coated with a polymer, preferably dextran, for the purpose to prevent formation of large-scale aggregates. The magnetic nanoparticles are superparamagnetic and are organized in chain-like structures that cause high dielectric losses of magnetic liquid in frequency range from 1 MHz to 30 MHz, which corresponds to frequencies commonly employed in RF-capacitive hyperthermia. Nanoparticles are combined into chains-like structures due to the interparticle coupling initiated by the magnetic dipole-dipole interaction. Necessary conditions for the formation of chains is the optimal ratio between the size of the magnetic core (iron oxide) and the shell thickness (e.g. dextran).

Another objective of the invention is associated with testing of mediator effect on the survival of malignant cells under the exposure to an ac electrical field.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other oral hygiene structures, methods and systems for carrying out the several purposes of the present disclosed device. It is important, therefore, that the claims be regarded as including such equivalent construction and methodology insofar as they do not depart from the spirit and scope of the present invention.

### BRIEF DESCRIPTION OF THE DRAWINGS

FIG. 1 is a diagram of RF capacitive applicator with phantom inserted between its electrodes.

FIG. 2 is a typical temperature profile of in vitro RF-capacitive hyperthermia treatment.

FIG. 3 is an X-ray diffraction (XRD) pattern of magnetic fluids N1 (top) and N4 (bottom).

FIG. 4 is a magnetisation curve of original and concentrated magnetic liquids (sample N4 and its concentrate).

FIG. 5 is a dielectric spectrum of magnetic liquids (samples N1-N7) compared with spectrum of distilled water: (a) real part and (b) imaginary part (dielectric losses) of complex permittivity.

FIG. 6. are results of in vitro RF-capacitive hyperthermia on (a) HaCaT and (b) HepG2 cells determined using MTT.

FIG. 7. are results of in vitro RF-capacitive hyperthermia on (a) HaCaT and (b) HepG2 cells determined using CFA.

### DETAILED DESCRIPTION OF THE PREFERRED EMBODIMENTS OF THE INVENTION

### Materials and Methods

Synthesis of Magnetic Liquids

Magnetic liquids were prepared by co-precipitation method in alkaline media starting from a mixture of FeCl₃x6H₂O and FeCl₂x4H₂O in twice distilled water; thereafter, an equal volume of stabilizer solution (dextran; 40 kDa) in distilled water was added to the iron salts solution. Aqueous ammonia was used as a precipitating agent. An approximately equal volume of NH₄OH (1 M) was added dropwise (1 drop/min) to an iron salts-polymer mixture under permanent stirring up to pH=10. After that, the solution was boiled for 15 minute at 60 degrees C. to form an almost black precipitate, which was dialyzed against unreacted iron salts during 48 hours with fourfold changing of water to maintain pH of about 6.5. Finally, the subsided iron-oxides particles were separated by ultracentrifugation and re-dispersed in distilled water by sonication. Substantially, seven types of magnetic liquids (N1-N7) with different structural and electromagnetic properties were prepared under the different Fe(III)/Fe(II) molar ratios (Table. 1 below).

TABLE 1
Characterisation of ferrofluid samples.

| | | | | c_{Fe} | dry | M_{S} | ε″ | ε″ |
|---|---|---|---|---|---|---|---|---|
| sam- | a₀ | d_{TEM} | d_{DLS} | (g | yield | (emu | [13.56 | [27.12 |
| ple | (Å) | (nm) | (nm) | l⁻¹) | (g l⁻¹) | g⁻¹) | MHz] | MHz] |
| N1 | 8.325 | 8-15 | 60 | 5.44 | 0.1 | 0.39 | 20 | 9.8 |
| N2 | 8.315 | 8-15 | 60 | 5.70 | 0.1 | 0.39 | 10 | 5.1 |
| N3 | 8.333 | 8-15 | 70 | 5.60 | 0.1 | 0.39 | 7 | 3.2 |
| N4 | 8.320 | 10-15 | 80 | 6.00 | 0.1 | 0.39 | 140 | 73.8 |
| N5 | 8.350 | 8-15 | 70 | 4.26 | 0.1 | 0.39 | 10 | 4.2 |
| N6 | 8.319 | 8-15 | 70 | 4.60 | 0.1 | 0.39 | 30 | 14.7 |
| N7 | 8.355 | 8-15 | 100 | 5.81 | 0.1 | 0.39 | 45 | 21.5 |

Characterization of Magnetic Liquids

The iron concentrations in magnetic liquids were determined by inductively coupled plasma-optical emission spectrometry (ICP spectrometer PRODIGY, Leeman Labs Inc., USA).

The structural characterization of magnetic liquids was carried out by Scanning Electron Microscopy (Superprobe microscope JEOL JXA 733, Japan) and Transmission Electron Microscopy (JEOL JEM 2000FX, Japan).

Light scattering analysis with scanning range from 0.6 to 6,000 nm (Malvern HPPS5001, UK) was used for determination of average particles size and aggregates of magnetic liquids.

X-ray diffraction patterns of samples were obtained by using an X′Pert PRO X-ray diffraction meter with Co Kα radiation at λ=0.179026 nm. Lattice parameters were refined with software package (HighScore Plus, PANanalytical, Netherlands).

The transmission ⁵⁷Fe Mössbauer spectra of samples were recorded using Mössbauer spectrometer (Wissel, Germany) in a constant acceleration mode with a gamma-ray source ⁵⁷Co(Rh). The isomer shift values were related to metal α-Fe at room temperature. Mössbauer spectra were measured in zero fields at temperatures from 5 to 300 K.

Magnetization measurements were obtained using a Vibrating Sample Magnetometer (Lake Shore 7404, USA) in the field up to 10 kOe. Dielectric measurements of ferrofluids were carried out by impedance method in frequency range from 10 Hz to 100 kHz by LCR (Hioki 3522, Japan), whereas in RF-range (from 10 MHz-20 GHz), the open reflection method was performed by Agilent 2-Port PNA-L (Microwave Analyzer N5230A, USA). In last method, the sample is measured by immersing the open-ended coaxial probe into a liquid.

In vitro Effect of RF Hyperthermia

Cell Culture

To express the effect of mediator on the malignant cells, two different cell lines were used: Human immortalized non-tumorogenic keratinocyte cell line (HaCaT) in accordance with supplied by Cell Lines Service (Catalog No. 300493, Germany), and human Hepatocellular carcinoma cell line (HepG2) from ATCC (HB-8065). To cultivate the HaCaT cells the Dulbecco's Modified Eagle Medium—high glucose, added 10% fetal bovine serum and Penicillin/Streptomycin, 100 U/ml /100 μg/ml, respectively (PAA Laboratories GmbH, Austria) was used as the culture medium. HepG2 cells were cultivated in ATCC-formulated Eagle's Minimum Essential Medium (ATCC) added 10% fetal bovine serum, 2 mM L-glutamine and 50 μg/ml gentamycine (PAA Laboratories GmbH, Austria). For cell cultivation, Ultrapure Water Systems Simplicity (Milipore, USA), Centrifuges Refrigerated centrifuge Eppendorf 5702 R (Eppendorf, Germany) and CO2 Incubator Heracell 150i (Thermo Scientific, USA) were used.

Description of In-Vitro RF Hyperthermia Treatment

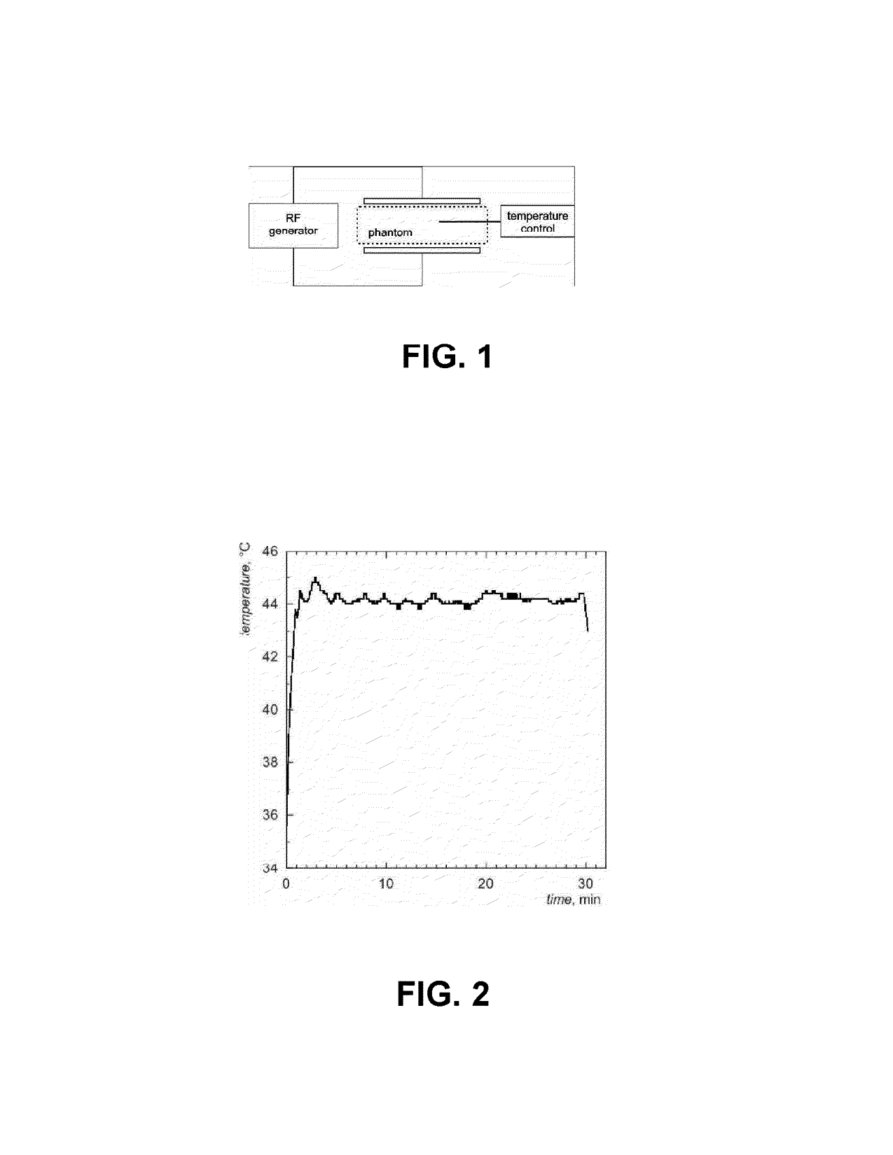

In order to test efficiency of prepared ferrofluid with regard to heat development under exposure to external dynamic electrical field and its consequential impact on a cell viability a series of tests have been carried out in a manner as follows. In vitro experiment was performed in Biological Safety Cabinet: HERAsafe KSP (Thermo Electron LED Gmbh, Germany). To find whether capacitive heating using mediator has additional effect on cell viability compared to standard heating procedure in water bath series of tests were performed. Refering now to FIG. 1, once a liquid sample of desired composition is prepared it is filled into a container of our own construction designed in a way securing a good electrical contact between applicator's electrodes and the volume of the sample. Subsequently the container is placed between two electrodes of the applicator ready for the treatment. A lab applicator of a capacitive type, EHY 110 (Oncontherm, Hungary), operating on a fixed frequency of 13.56 MHz was employed for the treatment. Temperature was measured using a pair of thermocouples. Treatment itself was designed in a following manner. Prior to in vitro testing, the certain magnetic liquid samples were filtered to remove the aggregates bigger than 0.2 μm (Milipore, USA). Then, liquid was mixed with cells in ratio 1:1 and 2:1, which corresponds to the following concentration of iron 3.5 g/l and 4.67 g/l in the tested volume. Total volume of the phantom was 8 ml, which was the amount of holder used for conduction of a hyperthermia session (FIG. 1).

Referring to FIG. 2, all samples were preheated to 37° C. Further heating was realized via capacitive applicator. In the first phase samples were rapidly heated from 37° C. up to a target temperature of 44° C. which was maintained for 30 minutes making sure it was not overshot (FIG. 2). For all tests same target temperature of 44° C. and treatment time of 30 minutes was used. Specific power employed during ramping was about 2.5 W/ml and 1.5 W/ml for steady state. Microscopic observation of cell culture before and after treatment was carried out by Inverted phase contrast microscope Olympus CKX 41 (Olympus, Japan), and Inverted fluorescent microscope Olympus IX51 (Olympus, Japan).

The effect of RF hyperthermia in the presence of mediator is presented on the series of tests performed on HaCaT and HepG2 cell lines. Viability was measured using the MTT assay (Invitrogen Corporation, USA) and colony forming assay. The absorbance in case of MTT assay was measured at 570 nm by Sunrise microplate absorbance reader (Tecan, Switzerland). With respect to different population doubling time of used cell line the distinct evaluation times were determined.

### Results

Properties of Iron-Oxide Based Magnetic Liquid

Magnetic liquid which has been developed is a stable dispersion of magnetic particles stabilized with a thin layer of a stabilizer in aqueous medium; it has high dielectric losses, which allow the material to be electrically heated and thus can be used as a mediator in RF hyperthermia, namely in RF-capacitive hyperthermia.

The high dielectric losses of said magnetic liquid result from its specific morphology, where magnetic particles are organized into chain-like structures, which are interconnected to form an infinite cluster.

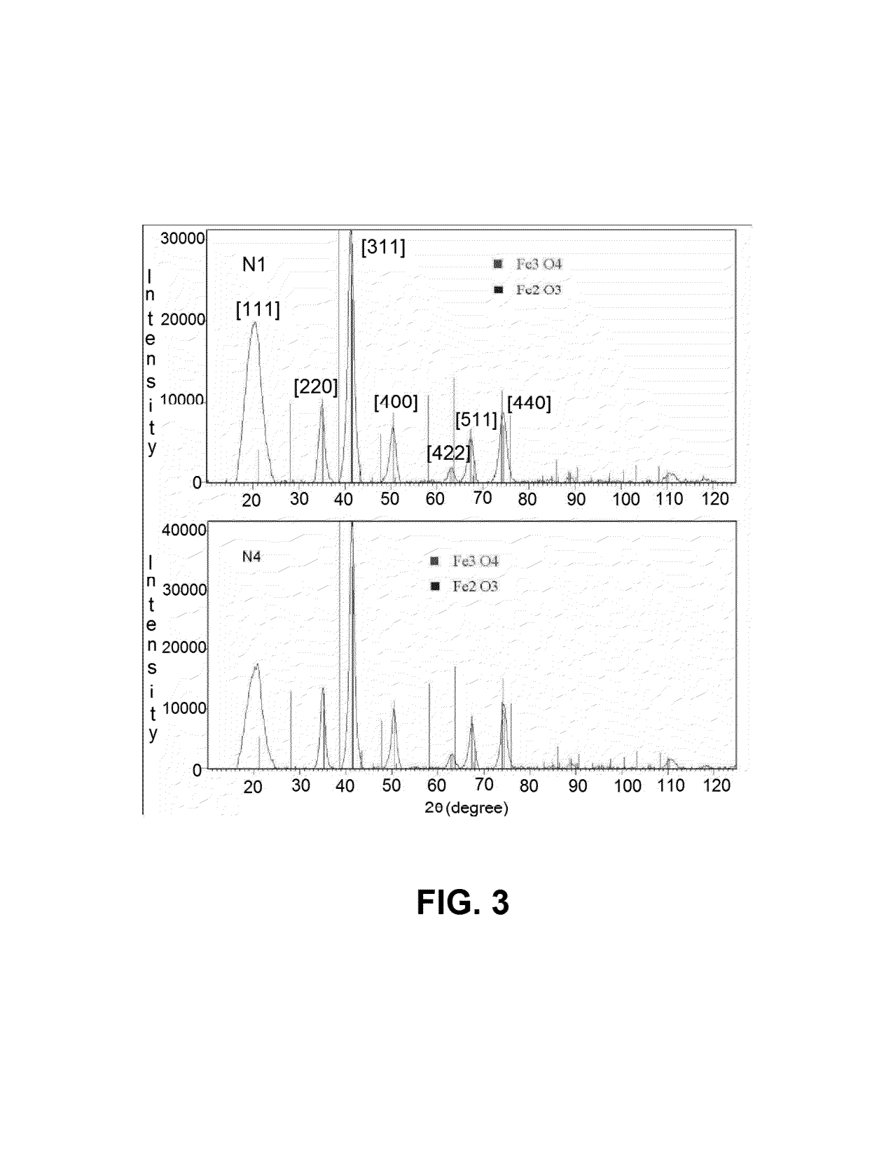

Refering now to FIG. 3, the magnetic particles are based on iron oxide nanoparticles with a size ranging from 8-10 nm in accordance with TEM images. Agreeable to X-Ray diffraction analysis, it is more probably maghemite (γ-Fe₂O₃) than magnetite (Fe₃O₄) as the value of lattice constant a0 is closer to that of crystalline maghemite (a0=8.345 Å) than to magnetite (a0=8.396 Å) (Table 1 above). However, sufficient minor differences of the X-ray patterns of magnetite and maghemite, such as an absence of 210 and 213 lines of maghemite, indicate that separated maghemite phase is not present as is shown in FIG. 3. This is likely to be caused by the partial oxidation and deviation from magnetite stochiometry. However, in accordance with results obtained by Mössbauer spectroscopy, it is maghemite. The mean hyperfine induction (hyperfine field) B_{hƒ} of the field spectrum taken at 5 K is B_{hƒ}=(51.98±0.24) T and isomer shift δ=(0.43±0.04) mm/s. These values are in a good agreement with B_{hƒ}for maghemite given in literature.

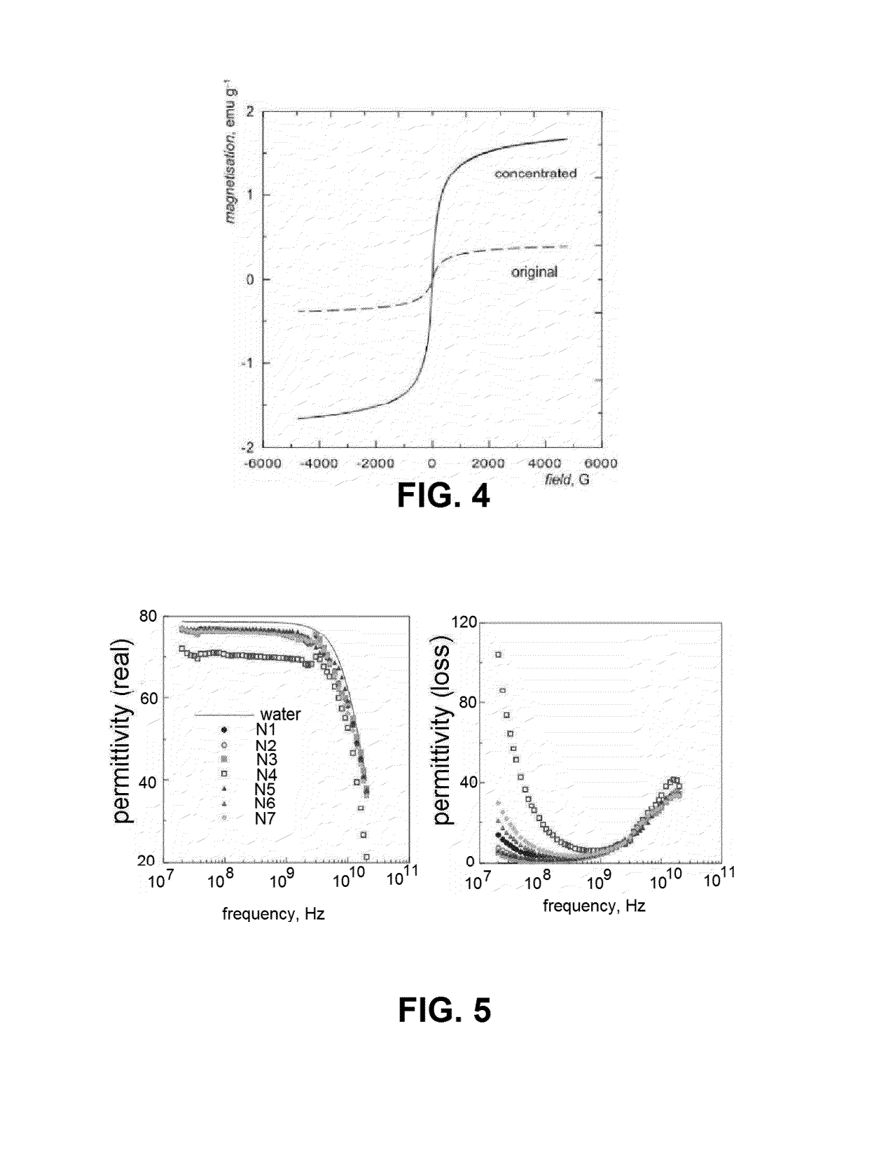

Iron-oxide nanoparticles are superparamagnetic as it was determined by Mössbauer spectroscopy and magnetostatic measurements. Mössbauer spectra recorded that the superparamagnetic relaxation occurs at temperatures above 270 K according to the depression of the magnetically split signal. Shown in FIG. 4, the magnetization curve follows the classical Langevin law. The recorded values of saturation magnetization (Mₛ), varying from 0.003 to 2 emu/g, depending on particle concentration, are rather negligible compared to bulk crystalline magnetite Ms=90 emu/g.

Nanoparticles have core-shell structure, where core consists of iron-oxides and shell comprising non-toxic, biodegradable and biocompatible polymer, namely dextran. The important factors in the choice of dextran are the optimum polar dextran-iron oxide interactions (mainly chelation and H-bonding) and favourable size of dextran chains which suggests precipitation within a polymer random coil structure. The well folded polymer creates confined space within which iron oxide crystal growth is limited and thus resulting in roughly spherical small particles with a size ranging from 8-15 nm where the polymer overlayer is about 1-3 nm. Each particle having magnetic moment leads to interparticle interaction and consequent creation of different types of aggregates (chain-like, globular, ring-like, etc.). According to the light scattering analysis, the mean aggregates size varies from 60 to 100 nm (Table 1 above). The microstructure of nanoparticle assembly is given by synthesis condition, namely polymer-stabiliser molecular weight (M_{w}) and its concentration, as well as corresponding molar ratio of ferrous chloride and ferric chloride. Thus, for 15 wt. % of dextran with M_{w}=40 kDa only molar ratio of Fe^{III/Fe}^{II} 1:1 yields particles organised into chain-like structures, which are interconnected to form an infinite cluster. Contrariwise, other molar ratio of _{Fe}^{III}/_{Fe}^{II} with same concentration of dextran does not lead to said self-organized structures.

In reference to FIG. 5, topological differences in magnetic liquids microstructure accordingly influence their dielectric properties. When the particles are arranged into chains forming an infinite cluster spanning throughout the volume of the sample, the dielectric losses are significantly higher than values recorded for other morphologies. The enhancement of imaginary part of complex permittivity was observed in frequency range below 1 GHz, namely from 1 MHz up to 30 MHz, the frequencies adopted in RF-capacitive hyperthermia (FIG. 5).

Therefore, magnetic liquid with chain-like structure (sample N4, Table 1) has been chosen as a mediator for investigation of hyperthermic effect in in vitro study.

In Vitro Effect of RF Hyperthermia on Human Cells

Results clearly indicate that usage of capacitive heating in the presence of mediator substantially contributes to cytotoxic effect of hypertermia treatment. As in vitro tests were performed, two methods of cell surviving based on different principles were employed so that their comparison could provide most relevant information to in vivo conditions. For clinical application it is important that observed effect increases with concentration of mediator. Even very low concentration of mediator has higher cytotoxic effect than only temperature effect alone (represented by heating in water bath). This additional effect is very important from the application point of view. The additional effect can be illustrated on the example of two different concentrations stated in Table 2 and 3 below. It is evident that unlike 3.5 g/l concentration the 4.67 g/l concentration leads to noticeable decrease in cells survival rate and consequent proliferation depending on used cell line and concentration of mediator.

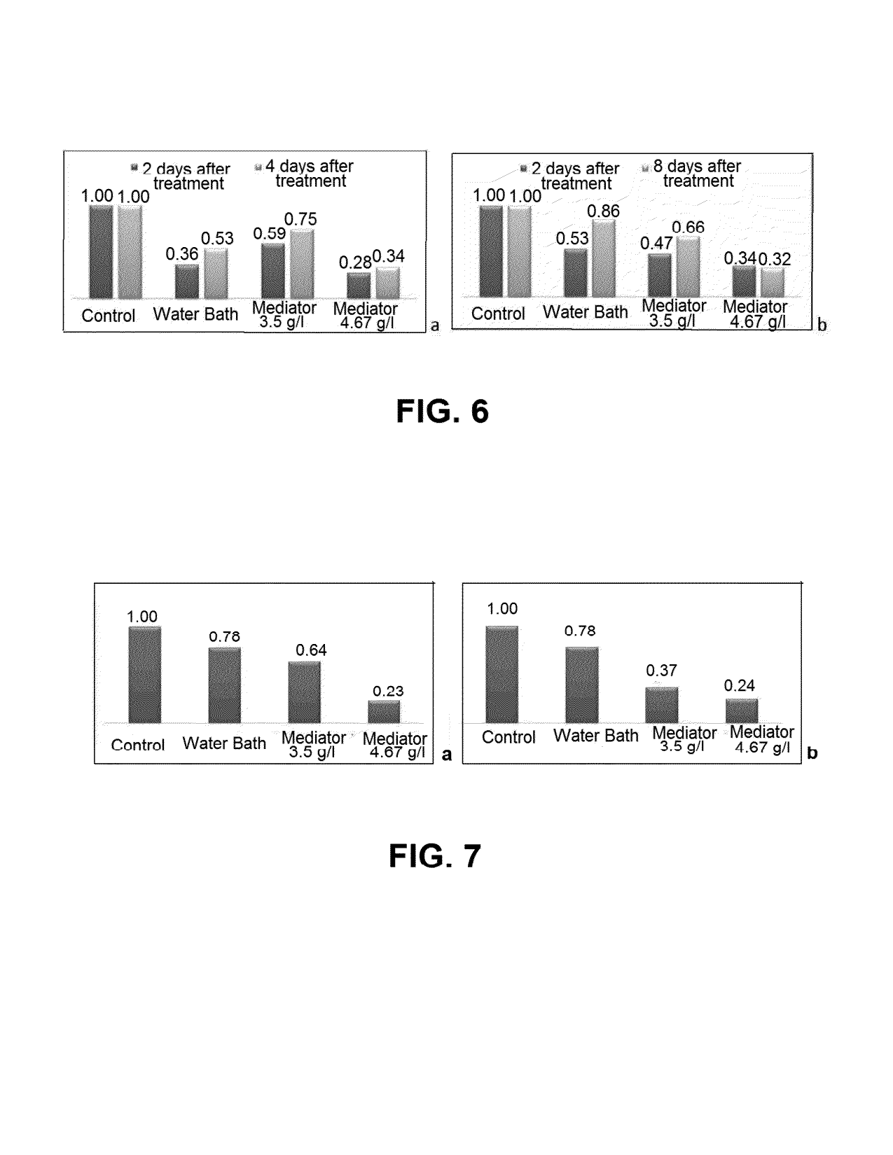

Refering now to FIG. 6 and FIG. 7, this effect was confirmed with both methods (tables 2 and 3 below; FIGS. 6 and 7). Unlike temperature effect, dependence of additional effect on mediator concentration enables one to adjust mediator concentration according to employed applicator and treated tumours.

TABLE 2
Results of in vitro tests for two cell lines determined using MTT.

| | no. days after | | | mediator | mediator |
|---|---|---|---|---|---|
| cell line | treatment | control | water bath | 3.5 g l⁻¹ | 4.67 g l⁻¹ |
| HepG2 | 2 | 1.00 | 0.53 | 0.47 | 0.34 |
| | 8 | 1.00 | 0.86 | 0.66 | 0.32 |
| HaCaT | 2 | 1.00 | 0.36 | 0.59 | 0.28 |
| | 4 | 1.00 | 0.53 | 0.75 | 0.34 |

TABLE 3
Results of in vitro tests for two cell lines determined using CFA.

| | | | mediator | mediator |
|---|---|---|---|---|
| cell line | control | water bath | 3.5 g l¹ | 4.67 g l¹ |
| HepG2 | 1.00 | 0.78 | 0.37 | 0.24 |
| HaCaT | 1.00 | 0.78 | 0.64 | 0.23 |

What is claimed is:

1. An electrically lossy magnetic liquid applicable for controlled tissue heating, preferably for tumour therapy by RF-hyperthermia, characterized by that it comprises of electrically conductive component formed by magnetic nanoparticles with core-shell structure where core size is between 10-15 nm and polymer shell thickness is between 1-3 nm; the said component is capable of subsequent dissipation of energy of electromagnetic radiation into heat resulting in selective and controlled heat treatment of defined extent of the tissue.

2. The magnetic liquid according to claim 1, characterized by that its core-shell structure is based on iron oxide-containing cores organized in chains-like structures and capable to generate heat by applying alternating electrical field due to dielectric losses.

3. The magnetic liquid according to claims 1 and 2, characterized by that the iron oxide-containing cores comprise magnetite, maghemite, or stoichiometric intermediate forms thereof.

4. The magnetic liquid according to claims 1 and 2, characterized by that the iron oxide-containing core is superparamagnetic.

5. The magnetic liquid according to claim 1, characterized by that its core-shell structure is formed in such a way that the said magnetic nanoparticles are encapsulated by biocompatible polymer shell.

6. The magnetic liquid according to claims 1, 2 and 5, characterized by that the biocompatible polymer is selected from polysaccharides, for example dextran.

7. A method of using of the magnetic liquid according to claim 1 and to any of claims 2-6 for controlled tissue heating, comprising:
accumulation of electrically lossy magnetic liquid in the tissue, the said liquid comprises chain-like structures, formed by iron-oxide core-shell nanoparticles and causing high dielectric losses of magnetic liquid under exposure to alternating electric field at the frequencies commonly employed in RF-capacitive hyperthermia, preferably at 13.56 MHz and 27.12 MHz;
application of alternating electrical field to generate heat in the tissue by excitation of electrically lossy magnetic liquid, thereby causing hyperthermia of the tissue.
